(19) United States
(12) Patent Application Publication
(10) Pub. No.: US 2023/0398269 A1
(43) Pub. Date: Dec. 14, 2023

(54) Breast Guard For Breast Pumping

(71) Applicant: ASO LLC, Sarasota, FL (US)

(72) Inventors: Edmund A. Sinda, Sarasota, FL (US); Brooke A. Hagerty, Sarasota, FL (US); Mira Dosen, Sarasota, FL (US)

(21) Appl. No.: 17/835,267

(22) Filed: Jun. 8, 2022

(51) Int. Cl. A61M 1/06

(52) U.S. Cl. CPC A61M 1/066

(57) ABSTRACT

A breast guard for use with a breast pump suction flange having an interior surface for placement against a wearer's breast and a liquid extraction port that extracts liquid from the wearer's breast includes a first liquid barrier in contact with the interior surface, a second liquid barrier in contact with the wearer's breast, and a third liquid barrier extending from the first liquid barrier to the second liquid barrier. The first liquid barrier, second liquid barrier, and third liquid barrier collectively form a liquid retention space that retains breast liquid therein. The breast guard may retain leakage and reduce irritation to the breast while breast pumping.

### FIELD

This relates to the field of breast pumping and, more particularly to, improving the comfort and retaining leakage during breast pumping sessions.

### BACKGROUND

Women use breast pumps to extract milk from their breasts for several reasons. Women who breastfeed their infants often breast pump to relieve pressure when the infant is not hungry. They also breast pump to have a store of breast milk available for bottle-feeding. With the number of working mothers on the rise, some women will pump to store milk for their partner or infant's caretaker to feed when the mother is unavailable. Women also breast pump to wean the infant from breastfeeding as they grow older. Breast milk may typically be stored up to four days if refrigerated or six to twelve months if frozen.

One common complication with breast pumping is that the milk can leak beyond the breast pump suction flange and onto one's skin during letdown. Letdown is a sudden release of milk during breast pumping or feeding. This occurs in waves multiple times throughout the pumping process until milk runs out. The first letdown cycle can release approximately one ounce of milk and decreases as pumping continues. As women differ in how much milk they produce, as well as their breast and nipple size, the suction flange sometimes does not properly connect to the skin and will spill out of the flange. Due to the spill, many women are placing a cloth or towel underneath the breast pump flange while pumping. The pumping process can last anywhere from 20 to 30 minutes and can have two to three letdowns.

Another cause of leaking is when the suction between the skin and the flange are detached after pumping is complete. The detachment can be abrupt and cause the milk to slide down the plastic or silicone of the flange and onto one's clothing. For working mothers or mothers generally pumping in public, the milk that leaks onto clothing during pumping can cause embarrassing stains.

Additionally, during pumping, mothers can also experience rubbing and friction of their areolas caused by a cyclical suction on/off process that extracts milk from the nipple. During suction, the nipple is stretched into the flange and can cause rubbing around the areola, which may lead to blisters or chafing. Currently, mothers who breast pump use balms in order to lubricate the breast, which can cause an oily film to remain on the flange, making it difficult to wash and sanitize.

### BRIEF SUMMARY

A first example of a breast guard that overcomes these drawbacks is for use with a breast pump suction flange having an interior surface for placement against a wearer's breast and a liquid extraction port that extracts liquid from the wearer's breast. When worn on the wearer's breast, the breast guard includes a first liquid barrier in contact with the interior surface, a second liquid barrier in contact with the wearer's breast, and a third liquid barrier extending from the first liquid barrier to the second liquid barrier. The first liquid barrier, second liquid barrier, and third liquid barrier collectively form a liquid retention space that retains breast liquid therein. This example of the breast guard may include one or more of the following features.

The liquid retention space may be beneath a nipple of the wearer.

The first liquid barrier may be adhered to the interior surface and the second liquid barrier may be adhered to the wearer's breast.

The third liquid barrier may be configured to permit the first liquid barrier and second liquid barrier to move independently of each other as the flange moves during suction on/suction off cycles of a breast pumping session.

The third liquid barrier may be collapsible.

The first liquid barrier, second liquid barrier, and third liquid barrier may be made of a flexible film of liquid impermeable material.

The second liquid barrier may include a washable low trauma adhesive for sealing against the wearer's breast.

A second example of a breast guard for use with a breast pump suction flange includes a breast contacting member including a flexible breast contacting member body that is sealable against a human breast. The breast contacting member body defines a first nipple receiving member. A flange contacting member includes a flexible flange contacting member body that is sealable against a breast pump suction flange. The flange contacting member body defines a second nipple receiving member. A collapsible member extends from the breast contacting member to the flange contacting member. The collapsible member forms a liquid barrier between the breast contacting member and flange contacting member. This example of the breast guard may include one or more of the following features.

The first nipple receiving member may include an opening defined by the breast contacting member body and the second nipple receiving member includes an opening defined by the flange contacting member body.

The collapsible member may be configured to permit the breast contacting member and flange contacting member to move independently of each other as the flange moves during suction on/suction off cycles of a breast pumping session.

The breast contacting member, flange contacting member, and collapsible member may collectively define a liquid retention space within the flange.

The breast contacting member body, flange contacting member body, and collapsible member may be made of at least one substantially liquid impermeable material.

The collapsible member may include a flexible sidewall made of substantially liquid impermeable material, the flexible sidewall being attached to breast contacting member and the flange contacting member.

A third example of a breast guard for use with a breast pump suction flange includes a breast contacting member including a breast contacting member body that is sealable against a human breast. The breast contacting member body defines a first nipple receiving member. A flange contacting member includes a flange contacting member body that is sealable against a breast pump suction flange. The flange contacting member body defines a second nipple receiving member. A collapsible member extends from the breast contacting member to the flange contacting member. The collapsible member is configured to permit the breast contacting member and flange contacting member to move independently of each other during suction on/suction off cycles of a breast pumping session. This example of the breast guard may include one or more of the following features.

The collapsible member may form a liquid barrier between the breast contacting member and flange contacting member when the breast contacting member body is against the human breast and the flange contacting member body is against the breast pump suction flange.

The first nipple receiving member may include an opening defined by the breast contacting member body and the second nipple receiving member may include an opening defined by the flange contacting member body.

The breast contacting member, flange contacting member, and collapsible member may collectively define a liquid retention space within the flange.

The breast contacting member body, flange contacting member body, and collapsible member may be made of at least one substantially liquid impermeable material.

The collapsible member may include a flexible sidewall made of substantially liquid impermeable material, the flexible sidewall being attached to breast contacting member and the flange contacting member.

A method of use includes reducing liquid leakage from a breast pump suction flange attached to a wearer's breast while pumping milk from the wearer's breast by forming a first liquid barrier against an interior of the flange, a second liquid barrier against the breast, and a third liquid barrier extending from the first liquid barrier to the second liquid barrier. The first liquid barrier, second liquid barrier, and third liquid barrier collectively form a liquid retention space that retains breast liquid within the flange. This method may include one or more of the following features.

The liquid retention space may be beneath a nipple of the wearer.

The first liquid barrier may be adhered to the interior and the second liquid barrier may be adhered to the wearer's breast.

The third liquid barrier may be configured to permit the first liquid barrier and second liquid barrier to move independently of each other as the flange moves during suction on/suction off cycles of a breast pumping session.

The third liquid barrier may be collapsible.

The first liquid barrier, second liquid barrier, and third liquid barrier may be made of a flexible film of substantially liquid impermeable material.

The third liquid barrier may include a washable low trauma adhesive for sealing against the wearer's breast.

The breast guard and method may include any combination of the aforementioned features.

### BRIEF DESCRIPTION OF THE DRAWINGS

FIG. 1 is a diagram of a conventional breast pump system in operation.

FIG. 2 is a side perspective view of an example of the breast guard.

FIG. 3 is a side plan view thereof seen from the side facing the breast contacting member.

FIG. 4 is a side plan view thereof seen from the side facing the flange contacting member.

FIG. 5 is a side plan view of a second example of the of the breast guard seen from the side facing the breast contacting member.

FIG. 6 is a side plan view thereof seen from the side facing the flange contacting member.

FIG. 7 is a side plan view of a third example of the of the breast guard seen from the side facing the breast contacting member.

FIG. 8 is a side plan view thereof seen from the side facing the flange contacting member.

FIG. 9 is a side plan view of a fourth example of the of the breast guard seen from the side facing the breast contacting member.

FIG. 10 is a side plan view thereof seen from the side facing the flange contacting member.

FIG. 11 is a diagram of the breast guard in a collapsed state being inserted into a breast pump suction flange.

FIG. 12 is a diagram of the breast guard installed in the breast pump suction flange.

FIG. 13 is a sectional view of the breast guard installed in the breast pump suction flange during a suction on cycle of a pumping session.

FIG. 14 is a sectional view of the breast guard installed in the breast pump suction flange during a suction off cycle of a pumping session.

FIG. 15 is a side perspective view of a fifth example of the breast guard.

FIG. 16 is a diagram of a sixth example of the breast guard installed in the breast pump suction flange.

### DETAILED DESCRIPTION OF EXAMPLES

This disclosure describes examples and features, but not all possible examples or features of the breast guard and related methods. Where a particular feature is disclosed in the context of a particular aspect or example, that feature can also be used, to the extent possible, in combination with and/or in the context of other features or examples. The breast guard may be embodied in many different forms and should not be construed as limited to only the examples and features described here.

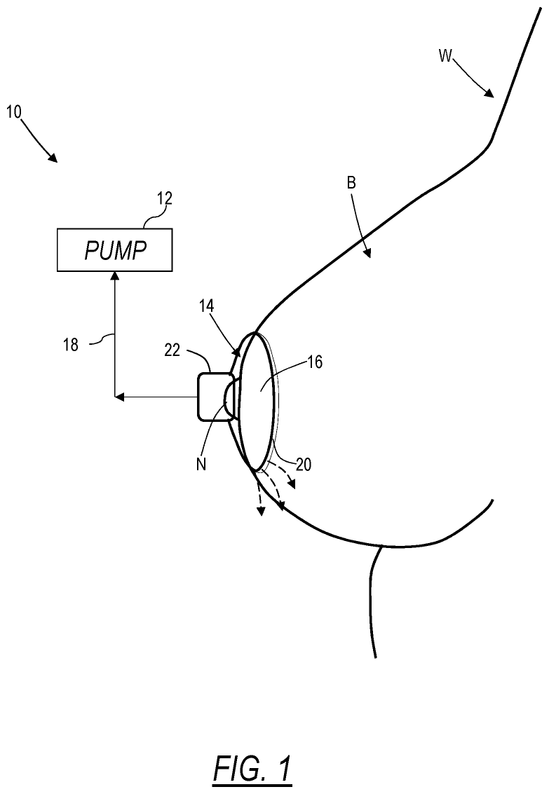

Referring to FIG. 1, a conventional breast pumping system 10 includes a pump 12 that applies suction to a suction flange 14 fitted tightly over the wearer's W breast B and extracts liquid from the breast B through a liquid conduit 18. The flange 14 includes a flange body 16 defining a flange perimeter 20 that encircles the wearer's nipple N. An extraction port 22 protruding from the flange body 16 proximal to the nipple N is attached to the conduit 18. In operation, the pump 12 applies suction via the conduit 18 through the extraction port 22 to the nipple N and liquid is extracted from the breast B. The problem with this construction is that liquid leakage, indicated by dashed-line arrows extending from the flange perimeter 20, often occurs.

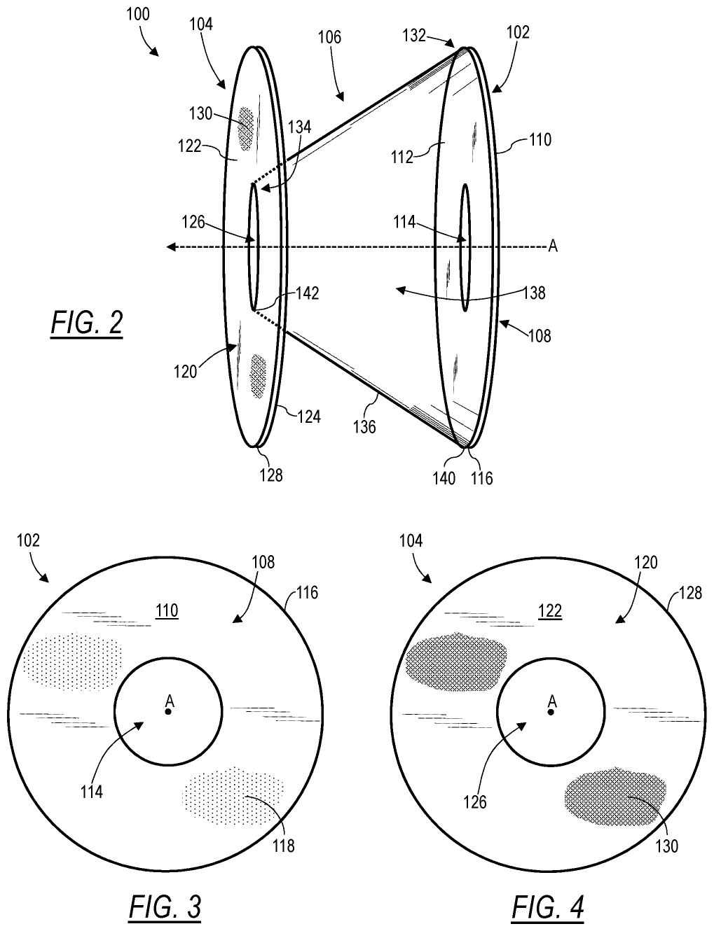

Referring to FIGS. 2-4, a first example of the breast guard 100 includes a breast contacting member 102, a flange contacting member 104 opposite the breast contacting member 102, and a collapsible member 106 extending from the breast contacting member 102 to the flange contacting member 104.

The breast contacting member 102 includes a breast contacting member body 108, including a breast contacting surface 110 and an interior surface 112 opposite the breast contacting surface 110. The breast contacting member body 108 defines a first nipple receiving opening 114, extending through the breast contacting member body 108 from the breast contacting surface 110 to the interior surface 112. The first nipple receiving opening 114 is arranged along a radial axis A passing through the breast guard 100. The breast contacting member body 108 extends to a breast contacting member body perimeter 116 that defines an outer terminal edge of the breast contacting member body 108.

As best seen in FIG. 3, the breast contacting surface 110 includes a skin contact adhesive 118 suitable for contacting the wearer's skin. The skin contact adhesive 118 may substantially cover the breast contacting surface 110 or it may be on discrete positions along the breast contacting surface 110 such as along the breast contacting member body perimeter 116 and/or circumscribing the first nipple receiving opening 114. The skin contact adhesive 118 is an adhesive suitable for contacting the skin and may be removable without substantially damaging the skin or causing pain to the wearer. The skin contact adhesive 118 may be washable, which means it can be washed with water but maintain its adhesive properties. This permits the breast guard 100 to be reused multiple times if desired. Examples of skin contact adhesive 118 materials include, for example, any adhesive suitable for use with materials that may contact human skin. Particular examples of skin contact adhesives 118 include, but are not limited to, low trauma adhesives such as low trauma acrylic adhesive, low trauma silicone adhesive, and the like.

The breast contacting member body 108 is made of flexible material capable of substantially conforming to the contour of a breast when applied thereto. The skin contact adhesive 118 holds the breast contacting member body 108 in place on the breast during pumping session. The skin contact adhesive 118 also forms a substantially liquid tight seal against the breast to prevent liquid leakage from the interior surface 112 to the breast contacting surface 110.

The breast contacting member body 108 material may be made of a substantially liquid impermeable material such as plastic, rubber, rubber-like, or plastic-like material. Examples of suitable materials include, but are not limited to, polyethylene, polyurethane, rubber, silicone, or the like. The substantially liquid impermeable material may be breathable if desired, meaning the material may permit some amount of water vapor to pass therethrough but will not substantially permit liquids to pass therethrough.

As best seen in FIGS. 2 and 4, the flange contacting member 104 includes a flange contacting member body 120, including a flange contacting surface 122 and an interior surface 124 opposite the flange contacting surface 122. The flange contacting member body 120 defines a second nipple receiving opening 126, extending through the flange contacting member body 120 from the flange contacting surface 122 to the interior surface 124. The second nipple receiving opening 126 is arranged along the radial axis A coaxial with the first nipple receiving opening 114. The flange contacting member body 120 extends to a flange contacting member body perimeter 128 that defines an outer terminal edge of the flange contacting member body 120.

As best seen in FIG. 4, the flange contacting surface 122 includes a flange contact adhesive 130 suitable for contacting the flange 14. The flange contact adhesive 130 may substantially cover the flange contacting surface 122 or it may be on discrete positions along the flange contacting surface 122 such as along the flange contacting member body perimeter 128 and/or circumscribing the second nipple receiving opening 126. The flange contact adhesive 130 is an adhesive suitable for contacting the flange 14 and may be removable without substantially damaging the flange 14. Examples of flange contact adhesive 130 materials include, for example, low trauma adhesives such as low trauma acrylic adhesive, low trauma silicone adhesive, and the like, or they may include other conventional adhesives.

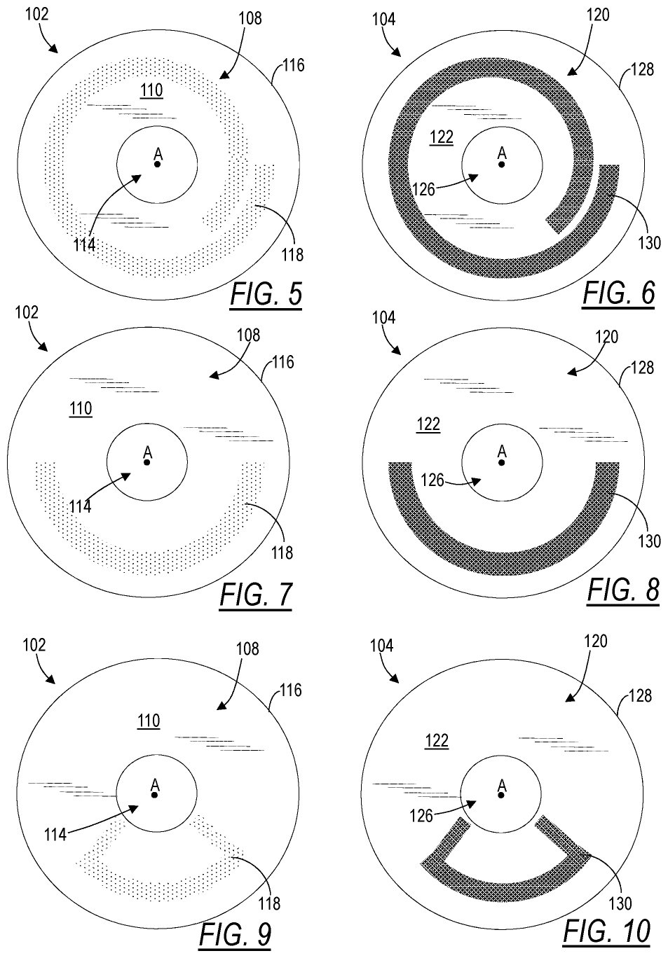

FIGS. 5-10 show several of many possible examples of how the skin contact adhesive 118 and flange contact adhesive 130 may be arranged.

In other examples, the skin contact adhesive 118 and/or flange contact adhesive 130 are separate from the breast guard 100, such as in the form of one or more adhesive tapes and/or a liquid adhesive that can be applied to the pertinent region.

The flange contacting member body 120 is made of flexible material capable of substantially conforming to the contour of the flange 14 when applied thereto. The flange contact adhesive 130 substantially holds the flange contacting member body 120 in place on the flange 14 during pumping session. The flange contact adhesive 130 also forms a substantially liquid tight seal against the flange 14 to prevent liquid leakage from the interior surface 124 to the flange contacting surface 122.

The flange contacting member body 120 material may be made of a substantially liquid impermeable material such as plastic, rubber, rubber-like, or plastic-like material. Examples of suitable materials include, but are not limited to, polyethylene, polyurethane, rubber, silicone, or the like.

The collapsible member 106 extends from the breast contacting member 102 to the flange contacting member 104 from a breast proximal side 132 to a flange proximal side 134 thereof. A sidewall 136 of the collapsible member 106 extends radially around the axis A and defines a liquid retention space 138 between the sidewall 136, breast contacting member interior surface 112, and flange contacting member interior surface 124. In this example the sidewall 136 tapers inwardly toward the flange contacting member interior surface 124, giving the liquid retention space 138 a frustoconical shape. The liquid retention space 138 receives and retains breast liquid leakage during a breast pumping session.

The sidewall 136 is attached to the breast contacting member 102 at a first seam 140. The first seam 140 may be formed by joining the sidewall 136 to the breast contacting member 102 via a plastic joining mechanism such as adhering, welding, melting, or the like. In other examples the sidewall 136 and breast contacting member 102 are unitary and formed from the same continuous piece of material.

The sidewall 136 is attached to the flange contacting member 104 at a second seam 142. The second seam 142 may be formed by joining the sidewall 136 to the flange contacting member 104 via a plastic joining mechanism such as adhering, welding, melting, or the like. In other examples the sidewall 136 and flange contacting member 104 are unitary and formed from the same continuous piece of material.

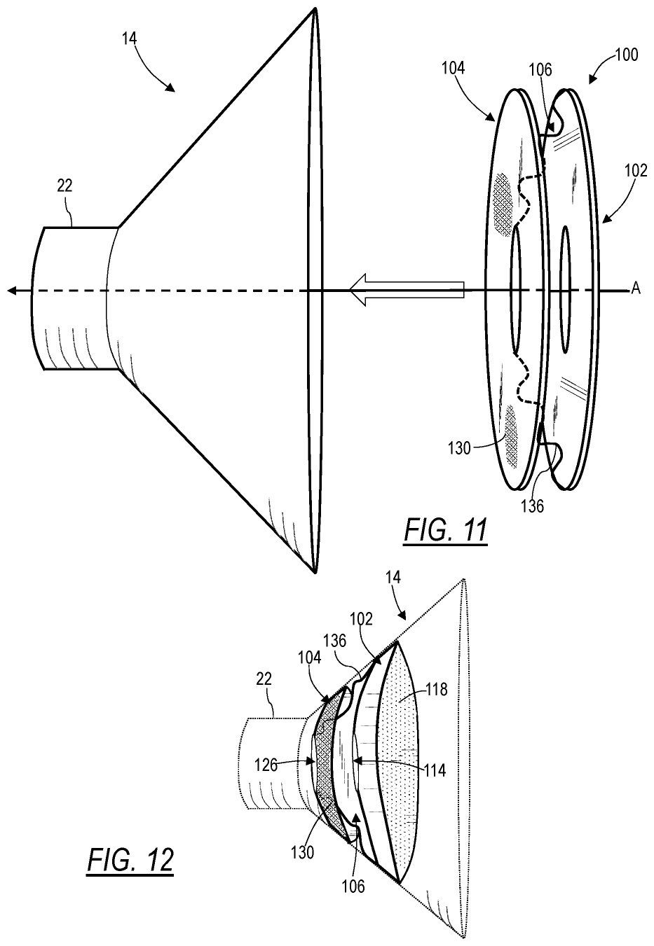

Referring to FIG. 11, the sidewall 136 is made of flexible material capable of collapsing to the point where the breast contacting member 102 and flange contacting member 104 are in contact or almost in contact with each other. The sidewall 136 material may be made of a substantially liquid impermeable material such as plastic, rubber, rubber-like, or plastic-like material. Examples of suitable materials include, but are not limited to, polyethylene, polyurethane, rubber, silicone, or the like. The substantially liquid impermeable material may be breathable if desired. The sidewall 136, breast contacting member 102, and flange contacting member 104 may be made of the same material or a different material. The sidewall 136, breast contacting member 102, and flange contacting member 104 may be made of a thin sheet of the selected liquid impermeable material, such as a sheet that is not more than 2 mm thick or 1 mm thick.

This collapsible function allows the breast guard 100 to be placed comfortably between the breast B and the inside surface of the suction flange 14 to capture leakage and provide a physical barrier between the flange 14 and the breast B to mitigate discomfort from rubbing to the wearer caused by the movement of the flange 14 during a pumping session.

Referring to FIGS. 11 and 12, the breast guard 100 may be inserted into the flange 14 such that the extraction port 22 is aligned along the axis A. The flange contact adhesive 130 is pressed against the flange 14 to secure the flange contacting member 104 to the inside of the flange 14. The breast (not shown) may be inserted into the flange 14 and pressed against the skin contact adhesive 118 to secure the breast B to the flange 14. Alternatively, the breast guard 100 may first be attached to the breast B and the flange 14 subsequently adhered to the flange contact adhesive 130.

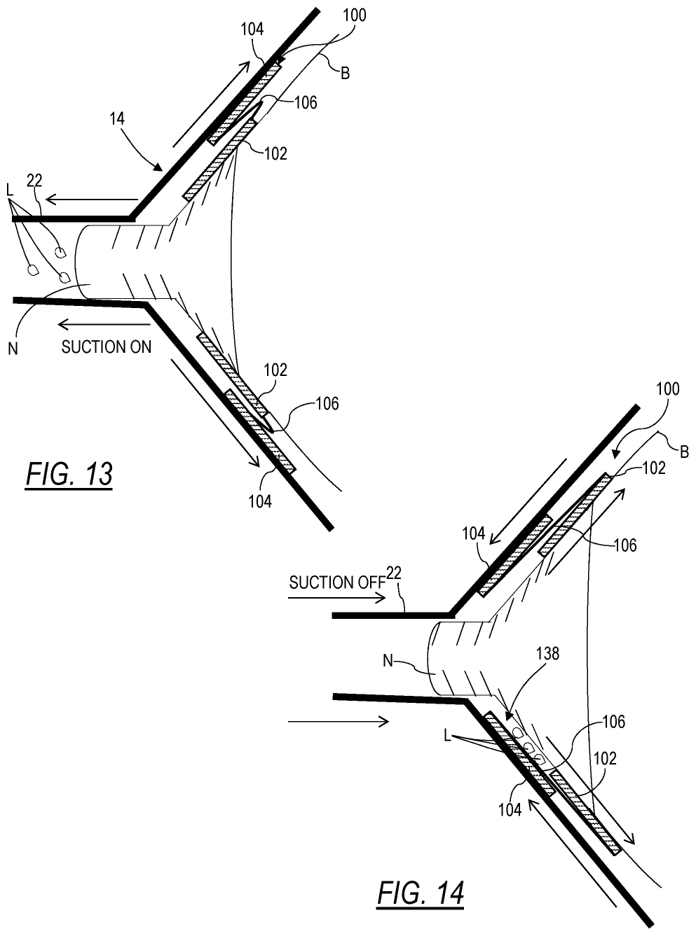

FIGS. 13 and 14 are sectional views of the breast guard 100 worn on a breast B and installed in the flange 14 during a breast pumping session. The breast pump 12 typically operates in suction on/suction off cycles, which generates the pumping action. These suction on/off cycles cause the flange 14 to move back and forth on the breast B, which can irritate the wearer's skin. FIG. 13 represents a suction on cycle and FIG. 14 represents a suction off cycle.

An advantageous feature of the breast guard 100 is that it permits the flange contacting member 104 and breast contacting member 102 to move independently of each other during the suction on/off cycles, but while the breast guard 100 is still able to capture liquid leakage from the breast B in the liquid retention space 138. Because the sidewall 136 is collapsible, the sidewall 136 is able to extend and compress as the flange contacting member 104 and breast contacting member 102 move independently of each other during the suction on/off cycles of a pumping session. This function reduces direct contact between the flange 14 and the breast B, which mitigates irritation to the breast B that might be caused by the flange 14 rubbing against it during suction on/off cycles. The breast guard 100, therefore, provides a physical barrier between the breast B and the flange 14.

Another advantageous feature of the breast guard 100 is that it provides a barrier to liquid L that leaks into the interior of the flange 14. As shown in FIG. 14, liquid L that leaks inside the flange 14 falls into the liquid retention space 138. The combination of the breast contacting member 102, flange contacting member 104, and collapsible member 106 surround the nipple N and substantially prevent the liquid L from leaking out the bottom of the flange 14 by retaining the liquid in the liquid retention space 138.

The breast guard 100 may also have many other designs besides the examples shown in FIGS. 2-14.

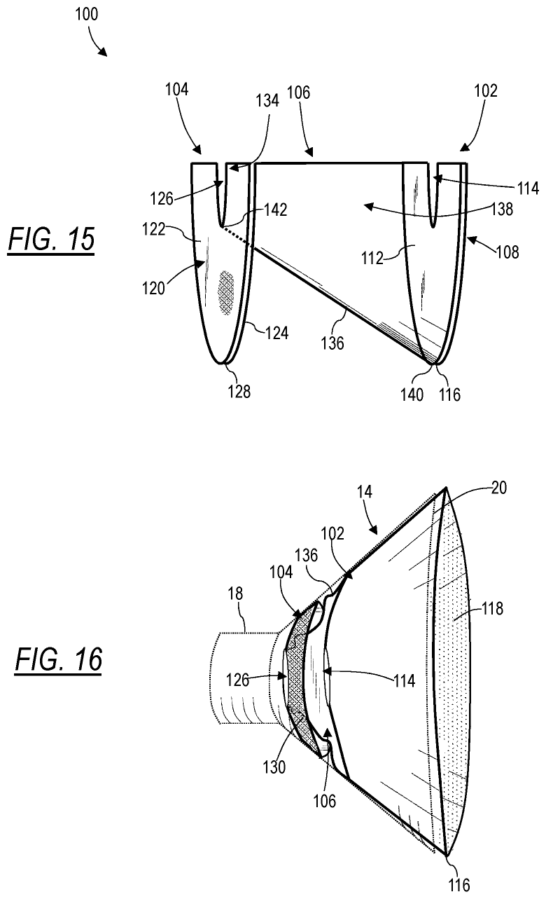

In other examples, such as the example in FIG. 15, the breast guard 100 may surround the nipple N by less than 360 degrees, such as from 5 degrees to 360 degrees. If the breast guard 100 does not surround the nipple N by 360 degrees, the first nipple receiving opening 114 and second nipple receiving opening 126 are not required to form a hole 360 degrees around the nipple N and may, therefore, form a cutout area for receiving the nipple N.

In other examples, such as the example in FIG. 16, the breast contacting member body perimeter 116 extends beyond the flange perimeter 20. This may provide additional adhesion to the breast B or additional surface area to reduce direct contact between the breast and suction flange 14.

The breast guard and related methods may be modified in many different ways without departing from the scope of what is claimed. The scope of the claims is not limited to the particular features and examples described above.

That which is claimed is:

1. A breast guard for use with a breast pump suction flange having an interior surface for placement against a wearer's breast and a liquid extraction port that extracts liquid from the wearer's breast, the breast guard comprising when worn the wearer's breast:
a first liquid barrier in contact with the interior surface;
a second liquid barrier in contact with the wearer's breast; and
a third liquid barrier extending from the first liquid barrier to the second liquid barrier;
wherein the first liquid barrier, second liquid barrier, and third liquid barrier collectively form a liquid retention space that retains breast liquid therein.

2. The breast guard of claim 1, wherein the liquid retention space is beneath a nipple of the wearer.

3. The breast guard of claim 1, wherein the first liquid barrier is adhered to the interior surface and the second liquid barrier is adhered to the wearer's breast.

4. The breast guard of claim 1, wherein the third liquid barrier is configured to permit the first liquid barrier and second liquid barrier to move independently of each other as the flange moves during suction on/suction off cycles of a breast pumping session.

5. The breast guard of claim 1, wherein the third liquid barrier is collapsible.

6. The breast guard of claim 1, wherein the first liquid barrier, second liquid barrier, and third liquid barrier are made of a flexible film of liquid impermeable material.

7. The breast guard of claim 1, wherein the second liquid barrier includes a washable low trauma adhesive for sealing against the wearer's breast.

8. A breast guard for use with a breast pump suction flange, the breast guard comprising:
a breast contacting member including a flexible breast contacting member body that is sealable against a human breast, the breast contacting member body defining a first nipple receiving member;
a flange contacting member including a flexible flange contacting member body that is sealable against a breast pump suction flange, the flange contacting member body defining a second nipple receiving member; and
a collapsible member extending from the breast contacting member to the flange contacting member, the collapsible member forming a liquid barrier between the breast contacting member and flange contacting member.

9. The breast guard of claim 8, wherein the first nipple receiving member includes an opening defined by the breast contacting member body and the second nipple receiving member includes an opening defined by the flange contacting member body.

10. The breast guard of claim 8, wherein the collapsible member is configured to permit the breast contacting member and flange contacting member to move independently of each other as the flange moves during suction on/suction off cycles of a breast pumping session.

11. The breast guard of claim 8, wherein the breast contacting member, flange contacting member, and collapsible member collectively define a liquid retention space within the flange.

12. The breast guard of claim 8, wherein the breast contacting member body, flange contacting member body, and collapsible member are made of at least one substantially liquid impermeable material.

13. The breast guard of claim 8, wherein the collapsible member includes a flexible sidewall made of substantially liquid impermeable material, the flexible sidewall being attached to breast contacting member and the flange contacting member.

14. A breast guard for use with a breast pump suction flange, the breast guard comprising:
a breast contacting member including a breast contacting member body that is sealable against a human breast, the breast contacting member body defining a first nipple receiving member;
a flange contacting member including a flange contacting member body that is sealable against a breast pump suction flange, the flange contacting member body defining a second nipple receiving member; and
a collapsible member extending from the breast contacting member to the flange contacting member, the collapsible member being configured to permit the breast contacting member and flange contacting member to move independently of each other during suction on/suction off cycles of a breast pumping session.

15. The breast guard of claim 14, wherein the collapsible member forms a liquid barrier between the breast contacting member and flange contacting member when the breast contacting member body is against the human breast and the flange contacting member body is against the breast pump suction flange.

16. The breast guard of claim 14, wherein the first nipple receiving member includes an opening defined by the breast contacting member body and the second nipple receiving member includes an opening defined by the flange contacting member body.

17. The breast guard of claim 14 wherein the breast contacting member, flange contacting member, and collapsible member collectively define a liquid retention space within the flange.

18. The breast guard of claim 14, wherein the breast contacting member body, flange contacting member body, and collapsible member are made of at least one substantially liquid impermeable material.

19. The breast guard of claim 14, wherein the collapsible member includes a flexible sidewall made of substantially liquid impermeable material, the flexible sidewall being attached to breast contacting member and the flange contacting member.

20. A method comprising reducing liquid leakage from a breast pump suction flange attached to a wearer's breast while pumping milk from the wearer's breast by forming a first liquid barrier against an interior of the flange, a second liquid barrier against the breast, and a third liquid barrier extending from the first liquid barrier to the second liquid barrier; the first liquid barrier, second liquid barrier, and third liquid barrier collectively forming a liquid retention space that retains breast liquid within the flange.

21. The method of claim 20, wherein the liquid retention space is beneath a nipple of the wearer.

22. The method of claim 20, wherein the first liquid barrier is adhered to the interior and the second liquid barrier is adhered to the wearer's breast.

23. The method of claim 20, wherein the third liquid barrier is configured to permit the first liquid barrier and second liquid barrier to move independently of each other as the flange moves during suction on/suction off cycles of a breast pumping session.

24. The method of claim 20, wherein the third liquid barrier is collapsible.

25. The method of claim 20, wherein the first liquid barrier, second liquid barrier, and third liquid barrier are made of a flexible film of substantially liquid impermeable material.

26. The method of claim 20, wherein the third liquid barrier includes a washable low trauma adhesive for sealing against the wearer's breast.
